(19) United States
(12) Patent Application Publication
(10) Pub. No.: US 2019/0110666 A1
(43) Pub. Date: Apr. 18, 2019

(54) INSERTION TOOL, INSERTION SYSTEM, AND DRIVE SOURCE

(71) Applicant: Olympus Corporation, Tokyo (JP)

(72) Inventor: Yutaka Masaki, Tokyo (JP)

(73) Assignee: Olympus Corporation, Tokyo (JP)

(21) Appl. No.: 16/216,427

(22) Filed: Dec. 11, 2018

Related U.S. Application Data
(63) Continuation of application No. PCT/JP2017/011170, filed on Mar. 21, 2017.

(30) Foreign Application Priority Data
Jun. 15, 2016 (JP) 2016-118891

(51) Int. Cl. A61B 1/00

(52) U.S. Cl. CPC A61B 1/00133; A61B 1/0016; A61B 1/00135; A61B 1/00128; A61B 1/00071

(57) ABSTRACT

An insertion tool includes a drive power transmitting mechanism disposed on a proximal-end portion of an insertion portion, for transmitting drive power to a driven member disposed on a distal-end side of the insertion portion. The drive power transmitting mechanism includes a gear train for transmitting drive power from a drive source that generates the drive power, and a joint mechanism for joining the drive source to the gear train to transmit the drive power from the drive source to the gear train and unjoining the drive source from the gear train.

### CROSS-REFERENCE TO RELATED APPLICATIONS

This application is a continuation application of PCT Application No. PCT/JP2017/011170 filed on Mar. 21, 2017, which in turn claim priority to the Japanese Patent Application No. 2016-118891 filed on Jun. 15, 2016 in Japan which is hereby incorporated by reference in its entirety.

### TECHNICAL FIELD

The present disclosure relates to an insertion tool for assisting an elongated tube to be inserted into a body using an electric drive source, an insertion system having such an insertion tool, and a drive source to be connected to an insertion tool when in operation.

### DESCRIPTION OF THE RELATED ART

US Patent Application Publication No. 2014/0330079 discloses an insertion system for assisting in inserting an insertion tool into and pulling the insertion tool from a lumen such as a large intestine or the like. With a mount tool suitably mounted on the outer circumference of an insertion portion of an insertion device of the insertion system, when an electric motor as an electric drive source is energized, the output shaft of the electric motor rotates the mount tool in a first direction or a second direction opposite the first direction. For example, when the mount tool is rotated in the first direction with respect to the insertion portion, the mount tool assists in inserting the distal end of the insertion portion into the lumen of a large intestine, for example, toward the deeper side thereof or in a direction away from the anus on account of friction between the inner circumferential surface of the lumen of the large intestine and the outer circumferential surface of the mount tool. With the mount tool suitably mounted on the insertion portion, when the mount tool is rotated in the second direction with respect to the insertion portion, the mount tool assists in pulling the distal end of the insertion portion with respect to the lumen of the large intestine toward the anus on account of friction between the inner circumferential surface of the lumen of the large intestine and the outer circumferential surface of the mount tool.

The electric drive source may not operate due to a fault, for example, even when supplied with electric power. Moreover, though the electric drive source itself is able to operate normally, the electric drive source may not operate appropriately due to a fault of a controller therefor. If the electric drive source does not operate appropriately while the distal end of the insertion portion with the mount tool mounted thereon is being placed in the lumen of a large intestine, for example, then it is necessary to pull the insertion portion and the mount tool out of the lumen by rotating the insertion portion and the mount tool in a predetermined direction or the second direction referred to hereinbefore about the axis of the insertion portion while pulling the insertion portion and the mount tool together toward the distal-end side. In particular, it is expected that the deeper the distal end of the insertion portion is in the lumen, the more tedious and time-consuming the pulling process would become.

### BRIEF SUMMARY OF EMBODIMENTS

The disclosed technology relates to an insertion tool that is capable of pulling an insertion portion with a mount tool mounted thereon easily out of a lumen or the like if an electric drive source fails to operate appropriately while an insertion system is in use, an insertion system having such an insertion tool, and a drive source to be connected to an insertion tool when in use.

An insertion tool according to an aspect of the present invention includes an insertion portion extending along a central axis, for being inserted into an examinee, and a drive power transmitting mechanism disposed on a proximal-end portion of the insertion portion, for transmitting drive power to a driven member disposed on a distal-end side of the insertion portion. The drive power transmitting mechanism includes a gear train for transmitting drive power from a drive source and a joint mechanism for joining the drive source to the gear train to transmit the drive power from the drive source to the gear train and unjoining the drive source from the gear train.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is a schematic view illustrating an insertion system according to a first embodiment.

FIG. 2A is a schematic view depicting a structure between a drive source disposed in a proximal-end portion of an insertion portion and a rotary unit disposed in the insertion portion in the insertion system according to the first embodiment.

FIG. 2B is a schematic view illustrating a bracket observed in the direction indicated by the arrow 2B in FIG. 2A, with a window being closed.

FIG. 2C is a schematic view illustrating the bracket observed in the direction indicated by the arrow 2B in FIG. 2A, with the window being open.

FIG. 3A is a schematic view illustrating a relay gear and a hub disposed on a rotational shaft of a drive power transmitting mechanism in the insertion system according to the first embodiment.

FIG. 3B is a schematic cross-sectional view taken along line 3B-3B of FIG. 3A.

FIG. 3C is a schematic view illustrating the rotational shaft of the drive power transmitting mechanism, the relay gear, and the hub observed in the direction indicated by the arrow 3C in FIG. 3A.

FIG. 4A is a schematic view illustrating a handle unit for use in place of the drive source in the insertion system according to the first embodiment.

FIG. 4B is a schematic view illustrating a joint shaft of the handle unit observed in the direction indicated by the arrow 4B in FIG. 4A.

FIG. 5 is a schematic view illustrating the manner in which an interlinked state between the drive source disposed in the proximal-end portion of the insertion portion and the drive power transmitting mechanism is canceled, and the rotational shaft of the drive power transmitting mechanism is fitted in a fitting hole in the joint shaft of the handle unit illustrated in FIG. 4A, in the insertion system according to the first embodiment.

FIG. 6A is a schematic view illustrating a drive source unit for use in place of the drive source in the insertion system according to the first embodiment.

FIG. 6B is a schematic view illustrating a joint shaft of the drive source unit observed in the direction indicated by the arrow 6B in FIG. 6A.

FIG. 7 is a schematic view illustrating the manner in which an interlinked state between the drive source disposed in the proximal-end portion of the insertion portion and the drive power transmitting mechanism is canceled, and the rotational shaft of the drive power transmitting mechanism is fitted in a fitting hole in the joint shaft of the drive source unit illustrated in FIG. 6A, in the insertion system according to the first embodiment.

FIG. 8 is a schematic view illustrating an insertion system according to a second embodiment.

FIG. 9 is a schematic view illustrating the manner in which a fitting hole in a joint shaft of a handle unit and a fitting hole in a joint shaft of a drive source unit can interchangeably be fitted over a rotational shaft of a drive power transmitting mechanism disposed in a proximal-end portion of an insertion portion in the insertion system according to the second embodiment.

FIG. 10 is a schematic view illustrating the manner in which the rotational shaft of the drive power transmitting mechanism is fitted in the fitting hole in the joint shaft of the drive source unit in the insertion system according to the second embodiment.

FIG. 11 is a schematic view illustrating the manner in which the rotational shaft of the drive power transmitting mechanism is fitted in the fitting hole in the joint shaft of the handle unit in the insertion system according to the second embodiment.

### DETAILED DESCRIPTION OF THE EMBODIMENTS

In the following description, various embodiments of the technology will be described. For purposes of explanation, specific configurations and details are set forth in order to provide a thorough understanding of the embodiments. However, it will also be apparent to one skilled in the art that the technology disclosed herein may be practiced without the specific details. Furthermore, well-known features may be omitted or simplified in order not to obscure the embodiment being described.

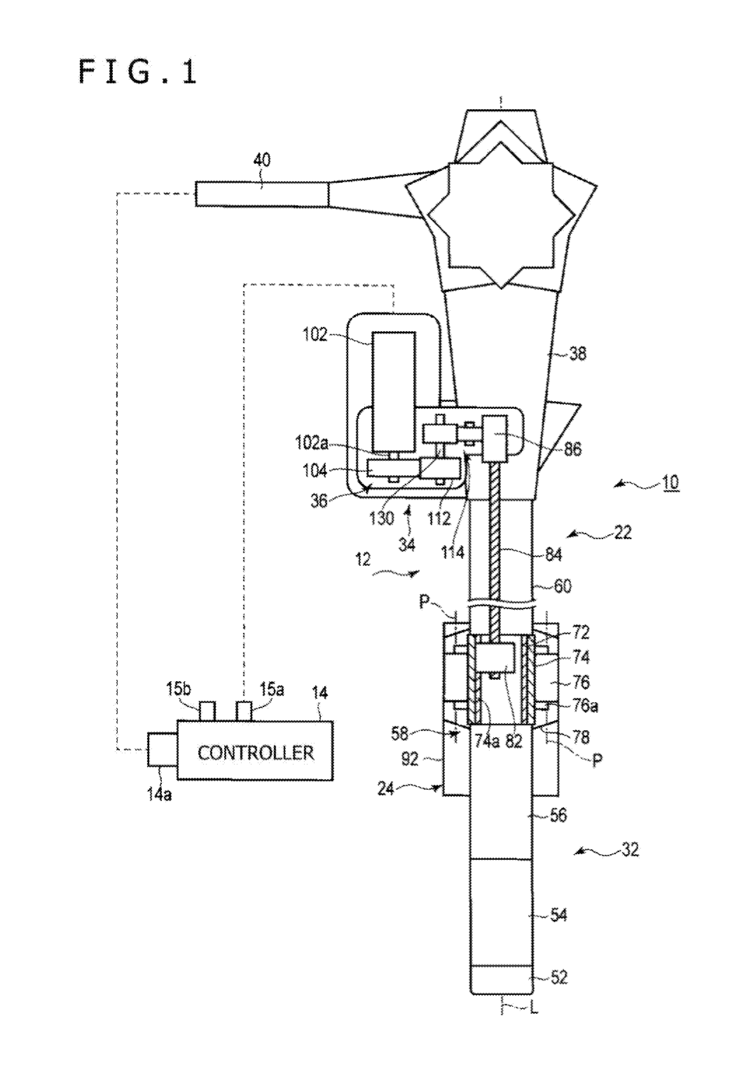

A first embodiment will be described hereinafter with reference to FIGS. 1 through 7.

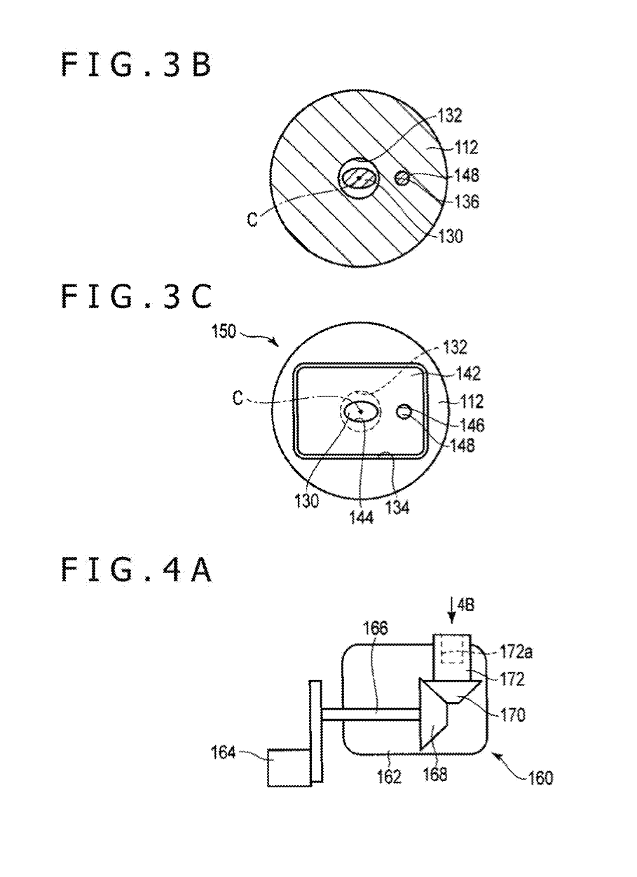

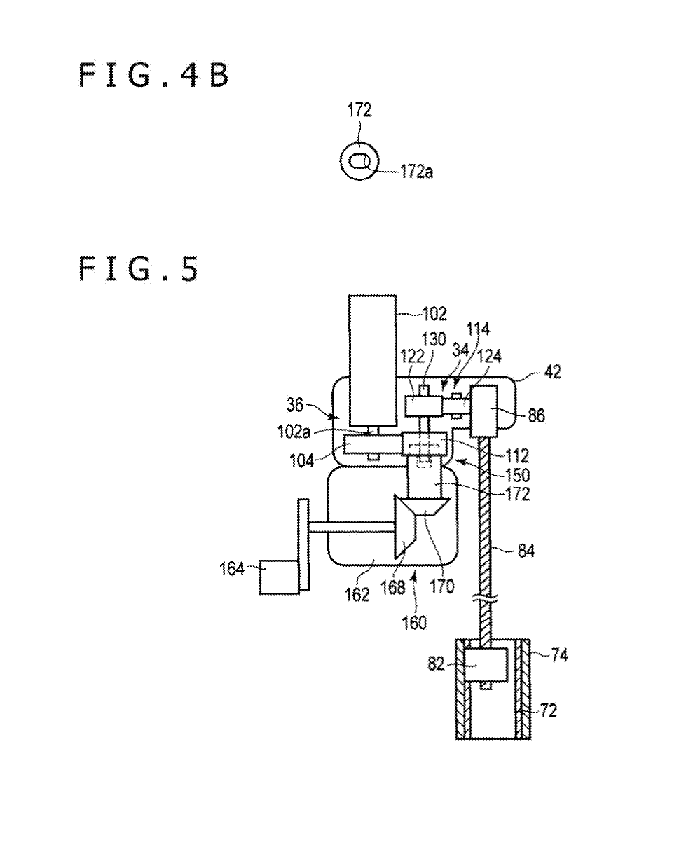

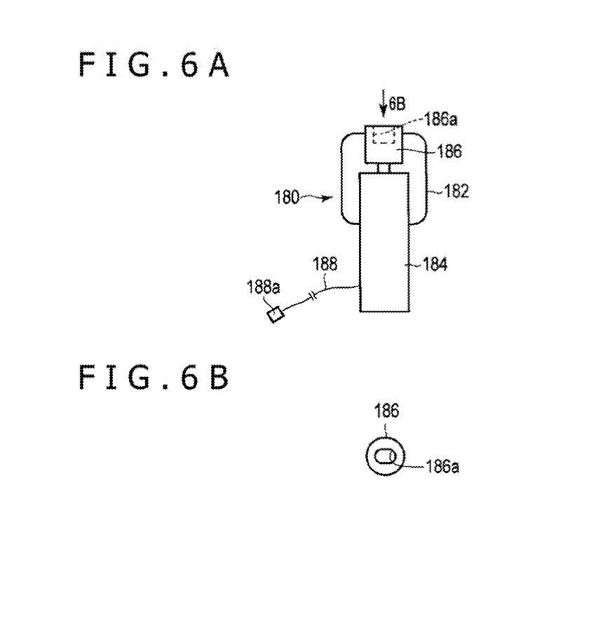

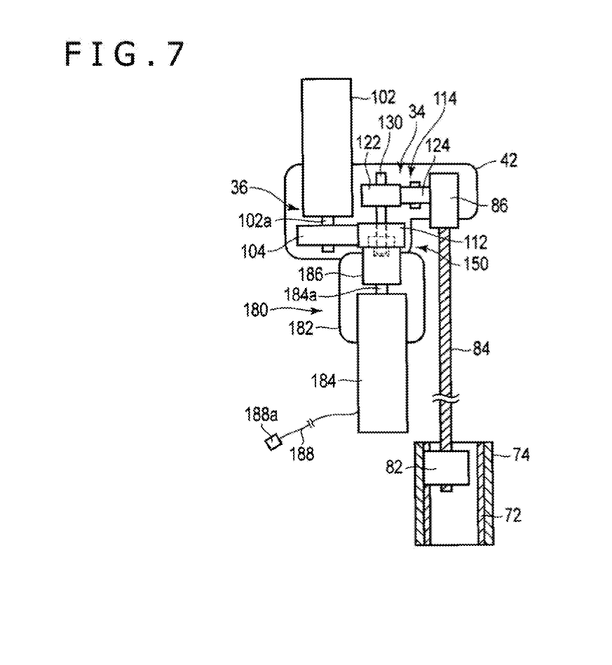

As illustrated in FIG. 1, an insertion system 10 according to the present embodiment has an insertion device 12 and a controller 14. Furthermore, the insertion system 10 has a handle unit 160 (see FIGS. 4A and 5) and/or an auxiliary drive source 180 (see FIGS. 6A and 7), to be describe hereinafter. The insertion device 12 will be described here as an endoscope by way of example. However, the insertion device 12 may be a catheter free of an illuminating optical system and/or an observation optical system.

The insertion device 12 has an insertion tool 22 and a mount tool 24, or spiral unit, mounted on the insertion tool 22. The mount tool 24 is rotatable together with a rotary unit 58, to be described hereinafter, of the insertion tool 22 about a central axis L of an insertion portion 32.

The insertion tool 22 has an insertion portion 32 extending along the central axis L and adapted to be inserted into an examinee, a drive power transmitting mechanism 34 mounted on a proximal-end portion of the insertion portion 32, a drive source 36, or electric drive source, for transmitting drive power to the drive power transmitting mechanism 34, a manipulator 38 mounted on the proximal-end portion of the insertion portion 32, and a universal cord 40 extending from the manipulator 38. The universal cord 40 is detachably connected to a main connector 14a of a controller 14.

According to the present embodiment, the controller 14 has a connector 15a to which a cable, not illustrated, for supplying electric power to the drive source 36 is detachably connected, and a spare connector 15b to which a cable, not illustrated, for supplying electric power to the auxiliary drive source 180 (see FIGS. 6A and 7) is detachably connected.

The insertion portion 32 has a distal-end portion 52, a bendable portion 54, a first flexible tube 56, a rotary unit 58, and a second flexible tube 60 in order from the distal-end side to the proximal-end side. The distal-end portion 52, the bendable portion 54, and the first flexible tube 56 may incorporate structural details of the insertion portion of a known endoscope. The second flexible tube 60 has a proximal end attached to the manipulator 38 as with the structure of a known endoscope.

The rotary unit 58 has, for example, a tubular base 72, a rotor 74, or driven member, disposed outwardly of the base 72 and having an internal gear 74a, a plurality of inner rollers 76, or driven members, movable around the central axis L of the rotor 74 through supports 76a disposed outwardly of the rotor 74 and rotatable about their own axes P parallel to the central axis L, and a tubular membrane 78 covering the outside of the rotor 74 and the inner rollers 76. The base 72 is attached in place between a proximal end of the first flexible tube 56 and a distal end of the second flexible tube 60. The membrane 78 has a distal end attached to the proximal end of the first flexible tube 56 and a proximal end attached to the distal end of the second flexible tube 60.

A drive gear 82 is disposed in the base 72. In the second flexible tube 60, a drive shaft 84 extends from a proximal-end side toward a distal-end side thereof. The drive shaft 84 has a central axis, or rotational shaft, substantially parallel to the central axis L of the insertion portion 32. An interlink gear 86 is attached to a proximal end, or one end, of the drive shaft 84. In the proximal-end portion of the insertion portion 32, i.e., in the manipulator 38 herein, the interlink gear 86 is held in mesh with an output gear 124 of a drive power transmitting gear train 114, to be described hereinafter, of the drive power transmitting mechanism 34.

The mount tool 24 has a tubular body 92 and is detachably mounted on an outer side of the membrane 78 of the rotary unit 58 by an appropriate structure. The mount tool 24 that is mounted on the outer side of the membrane 78 of the rotary unit 58 of the insertion portion 32 is rotatable around the central axis L, but is prevented from moving along the central axis L. Although not illustrated, the tubular body 92 should preferably have helical protrusions protruding radially outwardly from an outer circumferential surface from the tubular body 92 with respect to the central axis L. When the tubular body 92 of the mount tool 24 is rotated clockwise, or in a first direction, with respect to the insertion portion 32 as viewed from the manipulator 38 toward the distal-end side of the insertion portion 32, for example, the distal end of the insertion portion 32 is moved toward the deeper side of the lumen of a large intestine, or in a direction away from the anus on account of friction between the outer circumferential surface of the mount tool 24 and the inner circumferential surface of the lumen of the large intestine. When the tubular body 92 of the mount tool 24 is rotated counterclockwise, or in a second direction, with respect to the insertion portion 32 as viewed from the manipulator 38 toward the distal-end side of the insertion portion 32, the distal end of the insertion portion 32 is moved toward the user's side of the lumen of the large intestine, or the anus, on account of friction between the outer circumferential surface of the mount tool 24 and the inner circumferential surface of the lumen of the large intestine. The insertion system 10 may be used in an esophagus, for example, as a lumen.

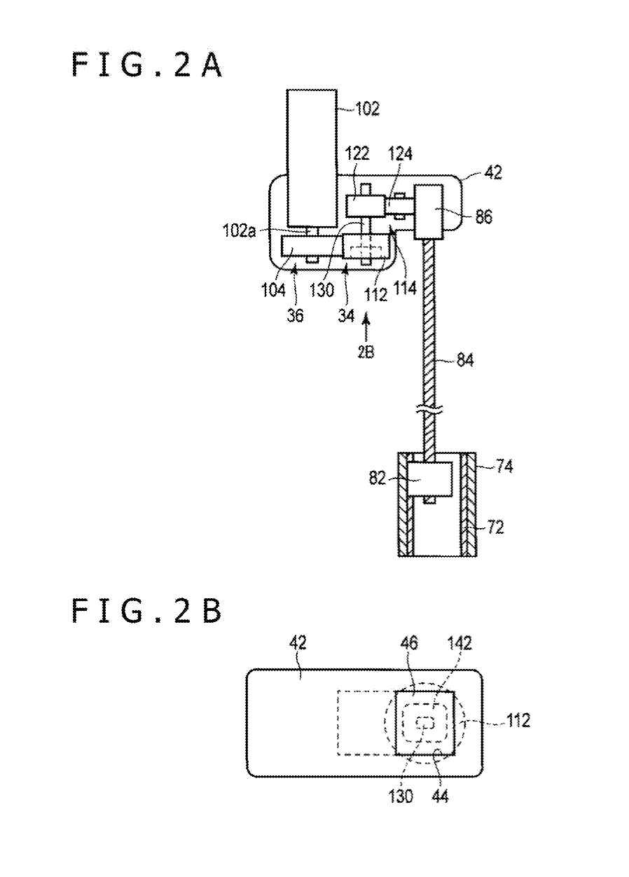

As illustrated in FIG. 2A, the drive power transmitting mechanism 34 and the drive source 36 are supported on a bracket 42, or storage case that is disposed on a proximal-end portion of the insertion portion 32, i.e., on the manipulator 38 herein, and that projects in a direction away from the central axis L. The drive source 36 has an electric motor 102 and an output gear 104 attached to an output shaft 102a of the electric motor 102. The electric motor 102 should preferably have a gear head, not illustrated, for adjusting the rotational torque of the output gear 104. The non-illustrated gear head should preferably be constructed as a speed reduction gear train.

The drive power transmitting mechanism 34 has a relay gear 112 and a drive power transmitting gear train 114. The relay gear 112 transmits drive power from the drive source 36 to the drive power transmitting gear train 114. The drive power transmitting gear train 114 should preferably be constructed as a speed reduction gear train.

The relay gear 112 meshes with the output gear 104 of the drive source 36. The drive source 36 and the drive power transmitting mechanism 34 transmit drive power, or rotational torque, of the output shaft 102a of the electric motor 102 through the output gear 104 and the relay gear 112 to the drive power transmitting gear train 114. The drive power transmitted to the drive power transmitting gear train 114 is transmitted through the interlink gear 86, the drive shaft 84, and the drive gear 82 to the rotor 74 that has the internal gear 74a. The rotor 74 rotates to cause the inner rollers 76 to revolve around the central axis L of the insertion portion 32 while rotating about their own axes on the supports 76a. Therefore, the tubular body 92 of the mount tool 24 on the outer side of the membrane 78 is rotated.

The drive power transmitting gear train 114 has an input gear 122 to which the drive power from the drive source 36 is input and an output gear 124 that outputs the drive power to the drive shaft 84. In FIG. 2A, the drive power transmitting gear train 114 is illustrated as having the input gear 122 and the output gear 124, though the number of gears thereof is not limited to 2, but may be greater. The drive power transmitting gear train 114 should preferably be of a structure for achieving appropriate speed reduction in the drive power after it is input to the input gear 122 until it is output to the output gear 124.

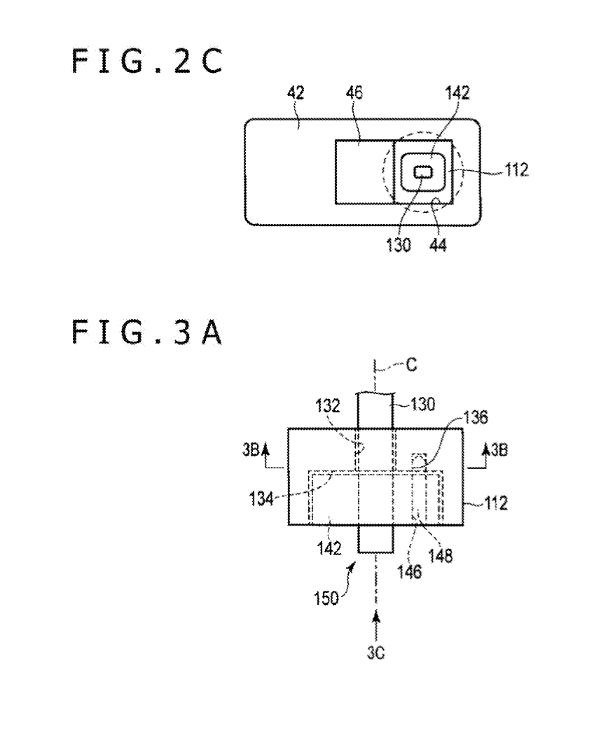

As illustrated in FIG. 2A, the relay gear 112 has a rotational shaft 130 that is shared by the input gear 122 of the drive power transmitting gear train 114. As illustrated in FIGS. 3B and 3C, the rotational shaft 130 should preferably be of a non-circular cross-sectional shape such as a substantially elliptical cross-sectional shape or the like. As illustrated in FIGS. 3A through 3C, the relay gear 112 has a through hole 132 defined therein at a position including its central axis C and having a size large enough to allow the rotational shaft 130 to rotate idly therein. In other words, the rotational shaft 130 is disposed on the central axis C of the relay gear 112. The relay gear 112 has a recess 134 defined therein at a position including its central axis C. The recess 134 is of a non-circular shape. The recess 134 is disposed opposite to a position facing the drive power transmitting gear train 114. A hub 142, or adapter, can engage in the recess 134. Specifically, the recess 134 can fit over the hub 142, or adapter. The hub 142 has a through hole 144 defined therein that extends along an outer circumferential surface of the rotational shaft 130.

When the electric motor 102 is energized while the hub 142 is being fitted in the recess 134, the rotational power is transmitted successively to the output gear 104, the relay gear 112, the hub 142, and the rotational shaft 130 in the order named. Therefore, with the hub 142 fitted in the recess 134, the relay gear 112 transmits the rotational drive power of the output gear 104 to the drive power transmitting gear train 114.

When the electric motor 102 is energized while the hub 142 is being removed from the recess 134, the rotational power is transmitted successively to the output gear 104 and the relay gear 112 in the order named. Therefore, the relay gear 112 rotates depending on the drive power of the drive source 36. However, even when the relay gear 112 rotates, the rotational shaft 130 remains as it is, i.e., remains non-rotated. Therefore, even when the output gear 104 is rotated while the hub 142 is being removed from the recess 134 in the relay gear 112, the relay gear 112 rotates idly with respect to the rotational shaft 130, so that no rotational drive power is transmitted to the rotational shaft 130 and the drive power transmitting gear train 114.

Consequently, a joint mechanism 150 to be described hereinafter is capable of switching the drive source 36 (the first drive source), and the drive power transmitting gear train 114 selectively into an interlinked state and a non-interlinked state. When the hub 142 engages the relay gear 112, it brings the drive source 36 and the drive power transmitting gear train 114 into the interlinked state, and when the hub 142 is detached from the relay gear 112, it brings the drive source 36 and the drive power transmitting gear train 114 into the non-interlinked state.

The relay gear 112 has an internally threaded hole 136 defined therein that is open into the recess 134. The hub 142 also has an internally threaded hole 146 defined therein that is coaxial with the internally threaded hole 136 of the recess 134 in the relay gear 112 when the hub 142 is fitted in the recess 134 in the relay gear 112. The hub 142 is fastened to the relay gear 112 by a set screw 148 or the like.

The relay gear 112, the rotational shaft 130, and the hub 142 make up a joint mechanism 150 for joining the drive source 36 to the drive power transmitting gear train 114 for transmitting the drive power from the drive source 36 to the drive power transmitting gear train 114 and releasing the drive source 36 from the drive power transmitting gear train 114. The joint mechanism 150 includes a joint shaft 172 of the handle unit 160, or manual drive source, and a joint shaft 186 of the auxiliary drive source 180.

The electric motor 102 may suddenly fail to operate due to a fault or the like. Specifically, the electric motor 102 may suddenly fail to operate while the insertion portion 32 with the mount tool 24 mounted thereon is being inserted into a body cavity. In such a case, according to the present embodiment, the handle unit 160 (see FIGS. 4A through 5) or the auxiliary drive source 180 (see FIGS. 6A through 7) can be used apart from the drive source 36.

FIG. 4A illustrates the handle unit 160 that is capable of outputting drive power to the drive power transmitting gear train 114, apart from the drive source 36. FIG. 4B is a view illustrating the joint shaft 172 of the handle unit 160 as viewed in the direction indicated by the arrow 4B in FIG. 4A. FIG. 5 illustrates the manner in which a fitting hole 172a in the joint shaft 172 of the handle unit 160 is fitted over the rotational shaft 130.

The handle unit 160 is used together with the insertion tool 22, and is able to transmit drive power from the joint shaft 172 through the drive power transmitting gear train 114 to the rotor 74 and the rollers 76.

As illustrated in FIGS. 4A through 5, the handle unit 160 has a housing 162, an input handle 164, an input rotational shaft 166, a first bevel gear 168, a second bevel gear 170, and a joint shaft 172, or joint portion, supported on the housing 162. The housing 162 should preferably be able to be fitted with the bracket 42 at a predetermined position thereon by a known appropriate structure.

The input handle 164 and the joint shaft 172 are supported on the housing 162. The first bevel gear 168 is integral with an end of the input rotational shaft 166. The input handle 164 is coupled to the other end of the input rotational shaft 166. The second bevel gear 170 is integral with an end of the joint shaft 172. The first bevel gear 168 and the second bevel gear 170 are held in mesh with each other. When drive power is manually input to rotate the input handle 164 about the axis of the input rotational shaft 166, the drive power is transmitted from the first bevel gear 168 to the second bevel gear 170, rotating the joint shaft 172 about its own axis.

If the number of teeth of the first bevel gear 168 is smaller than the number of teeth of the second bevel gear 170, then when rotation of the first bevel gear 168 causes the second bevel gear 170 and hence the joint shaft 172 to rotate, the rotational speed of the joint shaft 172 is made lower than the rotational speed of the input rotational shaft 166. At this time, the rotational torque of the second bevel gear 170 is made larger than the rotational torque of the first bevel gear 168. If the number of teeth of the first bevel gear 168 is larger than the number of teeth of the second bevel gear 170, then when rotation of the first bevel gear 168 causes the second bevel gear 170 and hence the joint shaft 172 to rotate, the rotational speed of the joint shaft 172 is made higher than the rotational speed of the input rotational shaft 166. At this time, the rotational torque of the second bevel gear 170 is made smaller than the rotational torque of the first bevel gear 168. The number of teeth of the first bevel gear 168 and the number of teeth of the second bevel gear 170 are set to appropriate values in view of the magnitude of manipulating power for rotating the input rotational shaft 166 of the handle 164.

As illustrated in FIGS. 4A and 4B, the joint shaft 172 has a fitting hole 172a defined therein. According to the present embodiment, the fitting hole 172a is of an elliptical shape and can fit over the rotational shaft 130. Therefore, the joint shaft 172 can be joined to the drive power transmitting gear train 114.

As illustrated in FIGS. 2B and 2C, the bracket 42 has a window 44 defined therein. The window 44 is defined at a position in which the hub 142 can be attached to and detached from the relay gear 112 and in which with the hub 142 detached from the relay gear 112, the fitting hole 172a in the joint shaft 172 can be fitted over the rotational shaft 130. As illustrated in FIG. 2B, the window 44 is normally closed by a slidable shutter 46. For installing the handle unit 160 or the auxiliary drive source 180 on the bracket 42 of the insertion system 10, the shutter 46 is shifted from a state in which it covers the window 44 (see FIG. 2B) to a state in which it releases the window 44 (see FIG. 2C). The shutter 46 may be broken to expose the relay gear 112 and the hub 142 through the window 44.

For installing the handle unit 160 on the bracket 42, the housing 162 is fitted over edges of the window 44. For installing the auxiliary drive source 180 on the bracket 42, a housing 182, to be described hereinafter, is fitted over edges of the window 44.

Instead of the window 44 defined in the bracket 42, the bracket 42 may have a portion separable therefrom. If the bracket 42 has a portion separable therefrom, then when that separable portion is separated, the hub 142 and the relay gear 112 are exposed, and with the hub 142 detached from the relay gear 112, the fitting hole 172a in the joint shaft 172 can be fitted over the rotational shaft 130. Alternatively, the bracket 42 may have a portion breakable therefrom. If the bracket 42 has a portion broken therefrom, then the hub 142 can be attached to and detached from the relay gear 112 and with the hub 142 detached from the relay gear 112, the fitting hole 172a in the joint shaft 172 can be fitted over the rotational shaft 130.

As illustrated in FIG. 5, if the fitting hole 172a in the joint shaft 172 of the handle unit 160 is to be fitted over the rotational shaft 130 of the drive power transmitting gear train 114, then the hub 142 and the relay gear 112 are exposed so that they can be accessed. Then, the set screw 148 is removed, and the hub 142 is detached from the relay gear 112 and the rotational shaft 130. The fitting hole 172a in the joint shaft 172 of the handle unit 160 now can be fitted over the rotational shaft 130 of the drive power transmitting gear train 114.

Although not illustrated, if the fitting hole 172a in the joint shaft 172 of the handle unit 160 is to be fitted over the rotational shaft 130 of the drive power transmitting gear train 114, then the housing 162 of the handle unit 160 should preferably be fitted with the bracket 42.

When the input handle 164 of the handle unit 160 is rotated, the joint shaft 172 is rotated about its own axis, rotating the rotational shaft 130. At this time, the relay gear 112 is idle, i.e., does not rotate, with respect to the rotational shaft 130, the electric motor 102 is prevented from generating electromotive forces.

Therefore, the drive power transmitting mechanism 34 transmits drive power, or rotational torque, of the joint shaft 172 of the handle unit 160 to the drive power transmitting gear train 114. The drive power transmitted to the drive power transmitting gear train 114 is transmitted through the interlink gear 86, the drive shaft 84, and the drive gear 82 to the rotor 74 that has the internal gear 74a. The rotor 74 rotates to cause the inner rollers 76 to revolve around the central axis L of the insertion portion 32 while rotating about their own axes on the supports 76a. Therefore, the tubular body 92 of the mount tool 24 on the outer side of the membrane 78 is rotated around the central axis L of the insertion portion 32. At this time, when the tubular body 92 of the mount tool 24 is rotated counterclockwise, or in the second direction, for example, with respect to the insertion portion 32 while the insertion portion 32 is being pulled toward the proximal-end side, the distal end of the insertion portion 32 is progressively moved toward user's side, or proximal-end side, on account of friction between the outer circumferential surface of the mount tool 24 and the inner circumferential surface of the lumen, for example.

When the joint mechanism 150 is holding the drive source 36 and the drive power transmitting gear train 114 in the non-interlinked state, the handle unit 160 can be joined as a drive source different from the drive source 36 to the joint mechanism 150. When the handle unit 160 is joined to the joint mechanism 150, the handle unit 160 and the drive power transmitting gear train 114 are held in the interlinked state. The insertion system 10 thus has another drive source 160 different from the drive source 36, which, when joined to the joint mechanism 150, is brought into the interlinked state with the drive power transmitting gear train 114 while the drive source 36 and the drive power transmitting gear train 114 are kept in the non-interlinked state by the joint mechanism 150.

With the insertion system 10 according to the present embodiment, therefore, even if the electric motor 102 fails to operate appropriately while the insertion system 10 is in use, inserting the insertion portion 32 with the mount tool 24 mounted thereon into a body cavity, the insertion portion 32 with the mount tool 24 mounted thereon can easily be pulled out of the body cavity by using the handle unit 160. Regardless of whether the electric motor 102 suffers a fault or the electric motor 102 fails to operate appropriately due to a fault of the controller 14, the insertion portion 32 with the mount tool 24 mounted thereon can easily be pulled out of the body cavity by using the handle unit 160.

FIG. 6A illustrates an auxiliary drive source 180 capable of outputting drive power to the drive power transmitting gear train 114. FIG. 6B is a view illustrating a joint shaft 186, to be described hereinafter, of the auxiliary drive source 180 observed in the direction indicated by the arrow 6B in FIG. 6A. FIG. 7 illustrates the manner in which the joint shaft 186 of the auxiliary drive source 180 is fitted over the rotational shaft 130.

The auxiliary drive source 180 is used with the insertion tool 22 and can transmit drive power from the joint shaft 186 through the drive power transmitting gear train 114 to the rotor 74 and the inner rollers 76.

As illustrated in FIGS. 6A through 7, the auxiliary drive source 180 has a housing 182, an electric motor 184, and the joint shaft 186, or joint portion. The joint shaft 186 has an elliptical fitting hole 186a defined therein. The housing 182 should preferably be able to be fitted with the bracket 42 at a predetermined position thereon by a known appropriate structure. The electric motor 184 can be connected to the spare connector 15b of the controller 14 by a connector 188a attached to a cable 188. The auxiliary drive source 180 may not necessarily be connected to the controller 14, but a battery may be disposed on the housing 182. The direction of rotation of an output shaft 184a of the electric motor 184 can be changed by a switch, not illustrated.

The electric motor 184 should preferably have a gear head, not illustrated, for adjusting the rotational torque of the joint shaft 186.

As illustrated in FIG. 7, if the fitting hole 186a in the joint shaft 186 of the handle unit 180 is to be fitted over the rotational shaft 130 of the drive power transmitting gear train 114, then the hub 142 is detached from the relay gear 112 and the rotational shaft 130 in the same manner as described hereinbefore. Then, the fitting holes 186a in the joint shaft 186 of the auxiliary drive source 180 can be fitted over the rotational shaft 130 of the drive power transmitting gear train 114. Therefore, the joint shaft 186 can be connected to the drive power transmitting gear train 114.

Although not illustrated, if the fitting holes 186a in the joint shaft 186 of the auxiliary drive source 180 can be fitted over the rotational shaft 130 of the drive power transmitting gear train 114, then the housing 182 of the auxiliary drive source 180 should preferably be fitted with the bracket 42.

Then, electric power is supplied to the electric motor 184 of the auxiliary drive source 180, the output shaft 184a is rotated, rotating the joint shaft 186 about its own axis to rotate the rotational shaft 130. At this time, since the relay gear 112 is idle, i.e., does not rotate, despite the rotation of the joint shaft 186, the electric motor 102 is prevented from generating electromotive forces.

Therefore, the drive power transmitting mechanism 34 rotates the tubular body 92 of the mount tool 24 on the outer side of the membrane 78 around the central axis L of the insertion portion 32, with the drive power, or rotational torque, of the joint shaft 186. At this time, when the tubular body 92 of the mount tool 24 is rotated counterclockwise, or in the second direction, for example, with respect to the insertion portion 32, the distal end of the insertion portion 32 is progressively moved toward user's side on account of friction between the outer circumferential surface of the mount tool 24 and the inner circumferential surface of the lumen, for example. At this time, the user does not need to manually move a handle such as the handle 164 of the handle unit 160.

When the joint mechanism 150 is holding the drive source 36 and the drive power transmitting gear train 114 in the non-interlinked state, the auxiliary drive source 180 can be joined as a drive source different from the drive source 36 to the joint mechanism 150. When the auxiliary drive source 180 is joined to the joint mechanism 150, the auxiliary drive source 180 and the drive power transmitting gear train 114 are held in the interlinked state. The insertion system 10 thus has another drive source 180 different from the drive source 36, which, when joined to the joint mechanism 150, is brought into the interlinked state with the drive power transmitting gear train 114 while the drive source 36 and the drive power transmitting gear train 114 are kept in the non-interlinked state by the joint mechanism 150.

With the insertion system 10 according to the present embodiment, therefore, even if the electric motor 102 fails to operate appropriately while the insertion system 10 is in use, inserting the insertion portion 32 with the mount tool 24 mounted thereon into a body cavity, the insertion portion 32 with the mount tool 24 mounted thereon can easily be pulled out of the body cavity by using the auxiliary drive source 180. In particular, if the electric motor 102 suffers a fault, the insertion portion 32 with the mount tool 24 mounted thereon can easily be pulled out of the body cavity by using the auxiliary drive source 180.

If the electric motor 102 fails to operate appropriately due to a fault of the controller 14, then the auxiliary drive source 180 of the type in which a battery, not illustrated, is disposed on the housing 182 may be used. Therefore, the insertion portion 32 with the mount tool 24 mounted thereon can easily be pulled out of the body cavity by using the auxiliary drive source 180.

As described hereinbefore, the insertion system 10 according to the present embodiment offers the following advantages:

If the electric motor 102 fails to operate appropriately, for example, it is not easy to rotate the tubular body 92 of the mount tool 24 illustrated in FIG. 1. According to the present embodiment, when the hub 142 is mounted in the relay gear 112, drive power is transmitted from the drive source 36 to the drive power transmitting mechanism 34. When the hub 142 is detached from the relay gear 112, the transmission of drive power from the drive source 36 to the drive power transmitting mechanism 34 is interrupted, and drive power can be transmitted to the drive power transmitting mechanism 34 using the manual handle unit 160, or drive source, or the electric auxiliary drive source 180. Therefore, even if the electric motor 102 of the drive source 36 suffers a fault, the rotational shaft 130 can be rotated to rotate the tubular body 92 of the mount tool 24. Even when the tubular body 92 of the mount tool 24 is present in a body cavity or the like, the distal end of the insertion portion 32 of the insertion tool 22 can be pulled out of a tube in the body cavity or the like. Instead of the electric motor 102, the controller 14 may suffer a fault. In this case, the distal end of the insertion portion 32 of the insertion tool 22 can easily be pulled out of a tube in a body cavity or like by using the manual handle unit 160, rather than by rotating the insertion portion 32 and the tubular body 92 of the mount tool 24 together around the central axis L to pull the distal end of the insertion portion 32 out of the body cavity. Therefore, the insertion system 10 according to the present embodiment can pull the insertion portion 32 out of a tube in a body cavity or the like by rotating the insertion tool 24 around the central axis L of the insertion portion 32, without taking the trouble to pull and rotate the insertion portion 32 and the mount tool 24 together.

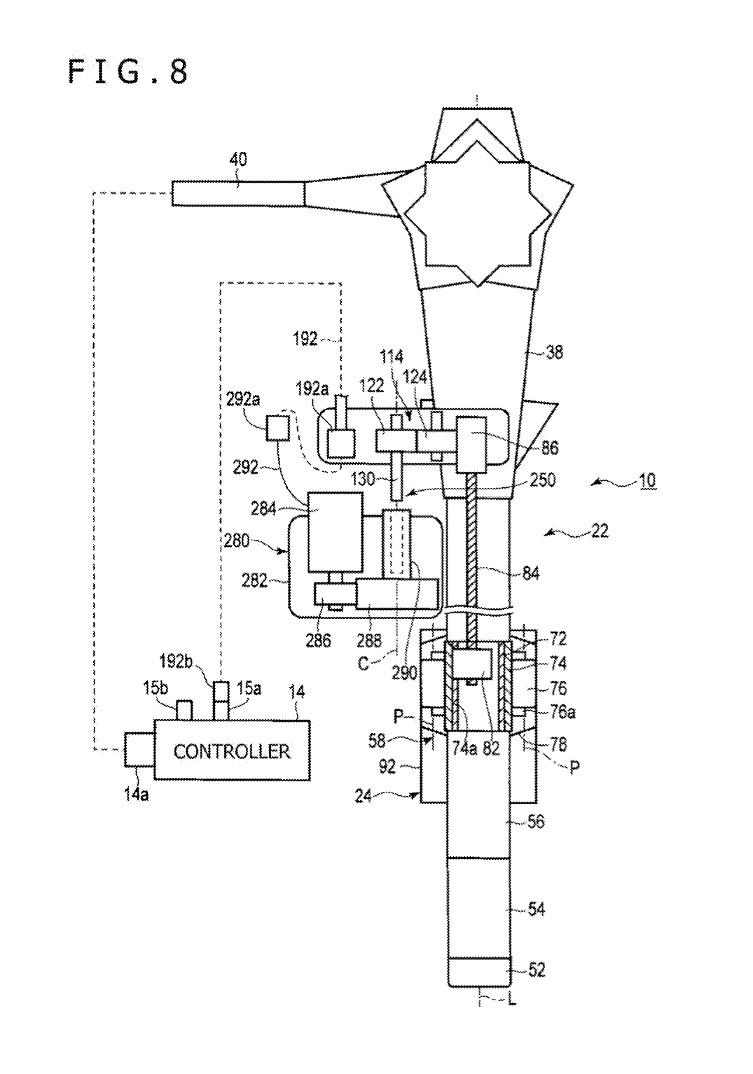

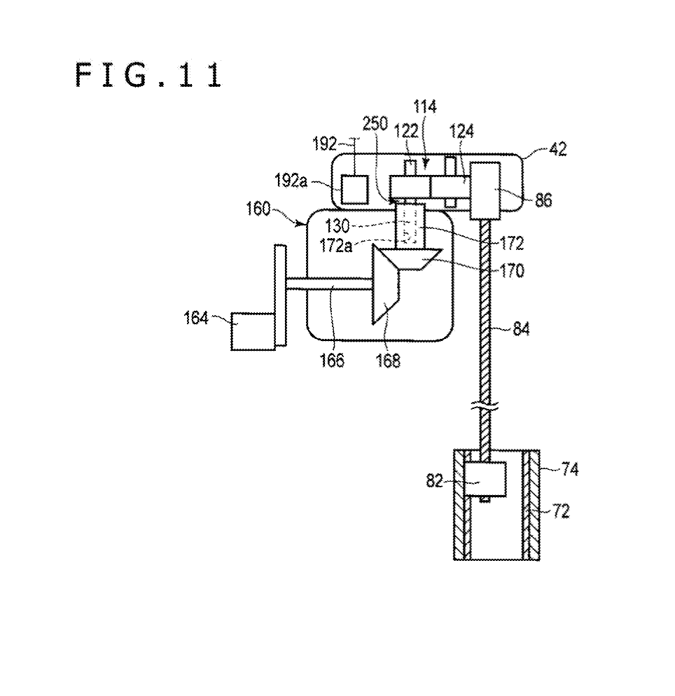

A second embodiment will be described hereinafter with reference to FIGS. 8 through 11. The present embodiment is a modification of the first embodiment, and those parts of the present embodiment which are identical to those described in the first embodiment are denoted by identical numeral reference and will not be described in detail hereinafter.

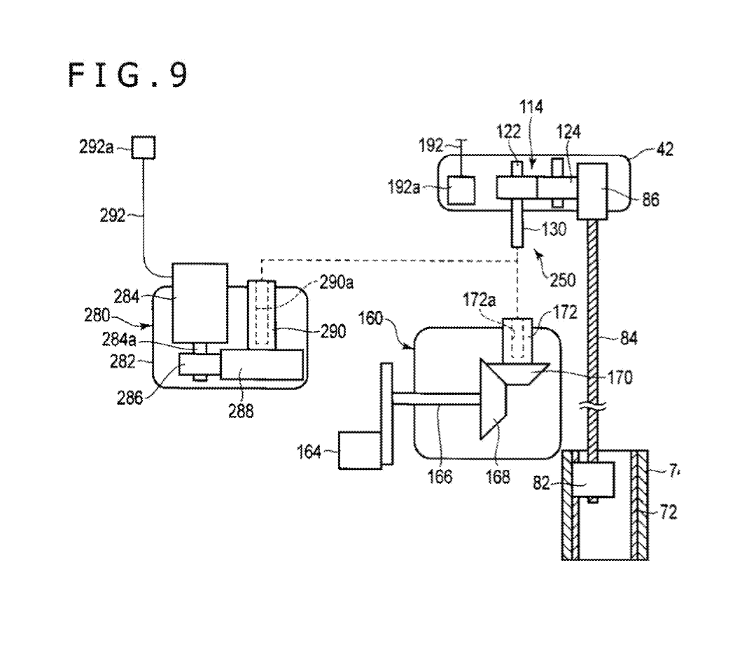

As illustrated in FIGS. 8 and 9, an insertion system 10 according to the present embodiment has an insertion device 12 and a controller 14. Furthermore, the insertion system 10 has a handle unit 160 (see FIGS. 4A, 9, and 11) and/or a drive source 280, or electric drive source (see FIGS. 8 through 10). The drive source 280 may be a single drive source or a plurality of drive sources.

According to the present embodiment, the drive source 280, or first electric drive source, is detachably mounted on the bracket 42. According to the present embodiment, the relay gear 112 (see FIG. 2A) described in the first embodiment is dispensed with. The electric drive source 280 and the handle unit 160 as a manual drive source are selectively coupled to the rotational shaft 130, which has an elliptical cross-sectional shape, for example, of the drive power transmitting gear train 114. Normally, the electric drive source 280 is coupled to the bracket 42. In the event of a fault of the electric drive source 280, the faulty drive source 280 is removed from the bracket 42, and a new spare electric drive source 280 is mounted on the bracket 42 and used. In the event of a fault of the electric drive source 280 or in the event of a fault of the controller 14, the joint shaft 172 of the handle unit 160 is coupled to the rotational shaft 130 and used. As described in the first embodiment, in the event of a failure of the controller 14, it is also preferable to use a drive source of the type in which it is supplied with electric power from a battery.

A terminal 192a of a cable 192 that is connected to the connector 15a of the controller 14 is attached to the bracket 42. A connector 292a, to be described hereinafter, of the drive source 280 can be connected to the terminal 192a.

As illustrated in FIGS. 8 through 10, the drive source 280 has a housing 282, an electric motor 284 having an output shaft 284a, an output gear 286, an interlink gear 288, and a joint shaft 290, or joint portion. The rotational shaft 130 and the joint shaft 290, or joint portion, make up a joint mechanism 250 capable of joining the drive source 280 and the drive power transmitting gear train 114 to transmit drive power from the electric motor 284 of the drive source 280 to the drive power transmitting gear train 114, and unjoining the drive source 280 from the drive power transmitting gear train 114. The joint mechanism 250 can join the handle unit 160 thereto for transmitting drive power from the handle unit 160 to the drive power transmitting gear train 114, and detach the handle unit 160 therefrom. The joint mechanism 250 can join the drive source 280 thereto for transmitting drive power from the drive source 280 to the drive power transmitting gear train 114, and detach the drive source 280 therefrom.

The housing 282 should preferably be able to be fitted with the bracket 42 at a predetermined position thereon by a known appropriate structure. The electric motor 284, the output gear 286, the interlink gear 288, and the joint shaft 290 are supported on the housing 282. The joint shaft 290 has a fitting hole 290a defined therein. According to the present embodiment, the fitting hole 290a is of an elliptical shape and can fit over the rotational shaft 130.

The connector 292a is connected to the electric motor 284 by a cable 292. The connector 292a can be connected to the terminal 192a attached to the bracket 42. The electric motor 284 can thus be energized by electric power from the controller 14.

According to the present embodiment, the bracket 42 has a window, not illustrated, defined therein. The window exposes the rotational shaft 130 therethrough. Consequently, the fitting hole 290a in the joint shaft 290 can be fitted over the rotational shaft 130.

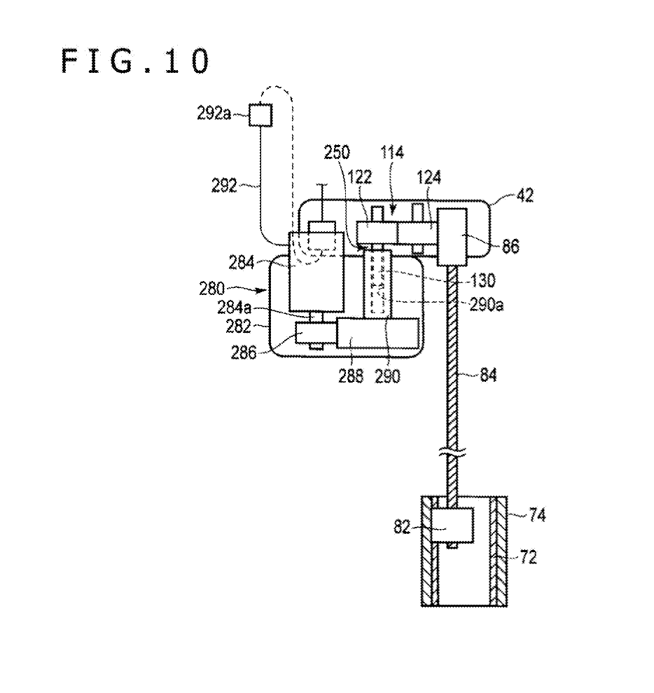

As illustrated in FIG. 10, the fitting hole 290a in the joint shaft 290 of the drive source 280 is fitted over the rotational shaft 130 of the drive power transmitting gear train 114. Although not illustrated, when the fitting hole 290a in the joint shaft 290 of the drive source 280 is fitted over the rotational shaft 130 of the drive power transmitting gear train 114, the housing 282 of the drive source 280 should preferably be fitted with the bracket 42.

When the output shaft 284a of the electric motor 284 of the drive source 280 is rotated, the joint shaft 290 is rotated about its own axis through the output gear 286 and the interlink gear 288, rotating the rotational shaft 130.

Therefore, the drive power transmitting mechanism 34 transmits drive power, or rotational torque, of the joint shaft 290 to the drive power transmitting gear train 114. The drive power transmitted to the drive power transmitting gear train 114 is transmitted through the interlink gear 86, the drive shaft 84, and the drive gear 82 to the rotor 74 that has the internal gear 74a. The rotor 74 rotates to cause the inner rollers 76 to revolve around the central axis L of the insertion portion 32 while rotating about their own axes on the supports. Therefore, the tubular body 92 of the mount tool 24 on the outer side of the membrane 78 is rotated around the central axis L of the insertion portion 32.

When the tubular body 92 of the mount tool 24 is rotated clockwise, or in the first direction, as viewed from the manipulator 38 toward the distal-end side of the insertion portion 32, for example, the distal end of the insertion portion 32 is moved toward the deeper side of the lumen of a large intestine, for example, or in a direction away from the anus on account of friction between the outer circumferential surface of the mount tool 24 and the inner circumferential surface of the lumen of the large intestine. When the tubular body 92 of the mount tool 24 is rotated counterclockwise, or in the second direction, as viewed from the manipulator 38 toward the distal-end side of the insertion portion 32, the distal end of the insertion portion 32 is moved toward the user's side of the lumen of the large intestine, or the anus, on account of friction between the outer circumferential surface of the mount tool 24 and the inner circumferential surface of the lumen of the large intestine. The insertion system 10 is thus able to assist in inserting the distal end of the insertion portion 32 into and pulling the distal end of the insertion portion 32 out of the tube with the drive power of the drive source 280.

With the insertion system 10 according to the present embodiment, if the electric motor 102 fails to operate appropriately while the insertion system 10 is in use, inserting the insertion portion 32 with the mount tool 24 mounted thereon into a body cavity, the drive source 280 is replaced with a new drive source 280 or with the handle unit 160.

For replacing the drive source 280, or first electric drive source, with a new drive source 280, or second electric drive source, the housing 282 of the drive source 280 is detached from the bracket 42, and the fitting hole 290a in the joint shaft 290 is released from the fitting engagement with the rotational shaft 130. Then, the housing 282 of the new drive source 280 is mounted on the bracket 42, and the fitting holes 290a in the joint shaft 290 is fitted over the rotational shaft 130 in the same manner as described hereinbefore. Consequently, in case the electric motor 284 suffers a fault and the controller 14 operates normally, a treatment or the like using the insertion device 12 can be continued as it is.

For replacing the drive source 280 with the handle unit 160, the housing 282 of the drive source 280 is detached from the bracket 42, and the fitting hole 290a in the joint shaft 290 is released from the fitting engagement with the rotational shaft 130. Then, the housing 162 of the handle unit 160 is mounted on the bracket 42, and the fitting holes 172a in the joint shaft 172 of the handle unit 160 is fitted over the rotational shaft 130.

The rotational shaft 130 and the joint shaft 172, or joint portion, make up a joint mechanism 250 capable of joining the handle unit 160, or drive source, and the drive power transmitting gear train 114 to transmit drive power from the manual handle 164 of the handle unit 160, or drive source, to the drive power transmitting gear train 114, and unjoining the handle unit 160, or drive source, from the drive power transmitting gear train 114.

When the input handle 164 of the handle unit 160 is rotated, the joint shaft 172 is rotated about its own axis, rotating the rotational shaft 130. Therefore, the drive power transmitting mechanism 34 transmits drive power, or rotational torque, of the joint shaft 172 to the drive power transmitting gear train 114. At this time, when the tubular body 92 of the mount tool 24 is rotated counterclockwise, or in the second direction, for example, with respect to the insertion portion 32, the distal end of the insertion portion 32 is progressively moved toward user's side on account of friction between the outer circumferential surface of the mount tool 24 and the inner circumferential surface of the lumen, for example.

With the insertion system 10 according to the present embodiment, therefore, even if the electric motor 284 fails to operate appropriately while the insertion system 10 is in use, inserting the insertion portion 32 with the mount tool 24 mounted thereon into a body cavity, the insertion portion 32 with the mount tool 24 mounted thereon can easily be pulled out of the body cavity by using the handle unit 160. Regardless of whether the electric motor 284 suffers a fault or the electric motor 284 fails to operate appropriately due to a fault of the controller 14, the insertion portion 32 with the mount tool 24 mounted thereon can easily be pulled out of the body cavity by using the handle unit 160.

With the insertion system 10 according to the present embodiment, furthermore, even if the electric motor 284 fails to operate appropriately while the insertion system 10 is in use, inserting the insertion portion 32 with the mount tool 24 mounted thereon into a body cavity, the insertion portion 32 with the mount tool 24 mounted thereon can easily be pulled out of the body cavity by using a new drive source 280 instead of the drive source 280 that has failed to operate. In particular, in the event of a fault of the electric motor 284, a treatment using the insertion system 10 can be continued as it is by using another new drive source 280.

If the electric motor 284 fails to operate appropriately due to a fault of the controller 14, then a new drive source 280 of the type in which a battery, not illustrated, is disposed on the housing 282 may be used. Therefore, the insertion portion 32 with the mount tool 24 mounted thereon can easily be pulled out of the body cavity by using the drive source 280.

In sum, one aspect of the disclosed an insertion tool comprises an insertion portion having respective opposed proximal-end portion and distal-end portion. The insertion portion extends along a central axis for being inserted into a body. A drive power transmitting mechanism is configured to be positioned on the proximal-end portion of the insertion portion for transmitting drive power to a driven member disposed on a distal-end side rather than the proximal-end portion. The drive power transmitting mechanism includes a gear train being used for transmitting drive power from a first drive source that is electrically driven. A joint mechanism being used for joining the first drive source to the gear train so as to transmit the drive power from the first drive source to the gear train and unjoining the first drive source from the gear train. And in a non-interlinked state in which the first drive source is unjoined from the gear train, a second drive source is joined to the joint mechanism to hold the gear train and the second drive source in an interlinked state.

The drive power transmitting mechanism is housed in a case disposed on the proximal-end portion of the insertion portion and the joint mechanism is capable to be exposed from the case. The joint mechanism includes a relay gear rotatable depending on the drive power from the first drive source. A hub is engageable with the relay gear for bringing the first drive source and the gear train into the interlinked state and detachable from the relay gear for bringing the first drive source and the gear train into the non-interlinked state. The gear train includes a rotational shaft disposed coaxially with a central axis of the relay gear. The rotational shaft transmits the drive power from the first drive source to the gear train when the hub engages the relay gear to bring the first drive source and the gear train into the interlinked state. The relay gear idles with respect to the rotational shaft when the hub is detached from the relay gear to bring the first drive source and the gear train into the non-interlinked state.

Another aspect of the disclosed technology is directed to an insertion system that in addition to the insertion tool, the second drive source that is different from the first drive source, the second drive source being joined to the joint mechanism to bring the second drive source and the gear train into the interlinked state when the joint mechanism holds the first drive source and the gear train in the non-interlinked state. The joint mechanism is capable of joining the first drive source to the joint mechanism for transmitting the drive power from the first drive source to the gear train and of unjoining the first drive source from the joint mechanism. The first drive source joined to the joint mechanism and the second drive source that is manually driven and joined to the joint mechanism while the first drive source is being unjoined. The first drive source joined to the joint mechanism and the second drive source that is electrically driven and joined to the joint mechanism while the first drive source is being unjoined. The gear train makes a speed reduction in the drive power transmitted from the joint mechanism to the driven member.

A further aspect of the disclosed technology is directed to a drive source used with the insertion tool and comprises a joint portion that is able to be joined to the gear train. The drive power is able to be transmitted from the joint portion through the gear train to the driven member. The drive source further comprises an electric motor supplied with electric power to generate the drive power transmitted from the joint portion through the gear train to the driven member. In addition, a manual handle manually operable to generate the drive power transmitted from the joint portion through the gear train to the driven member.

While various embodiments of the disclosed technology have been described above, it should be understood that they have been presented by way of example only, and not of limitation. Likewise, the various diagrams may depict an example schematic or other configuration for the disclosed technology, which is done to aid in understanding the features and functionality that can be included in the disclosed technology. The disclosed technology is not restricted to the illustrated example schematic or configurations, but the desired features can be implemented using a variety of alternative illustrations and configurations. Indeed, it will be apparent to one of skill in the art how alternative functional, logical or physical locations and configurations can be implemented to implement the desired features of the technology disclosed herein.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one”, “one or more” or the like; and adjectives such as “conventional”, “traditional”, “normal”, “standard”, “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more”, “at least”, “but not limited to” or other like phrases in some instances shall not be read to mean that the narrower case is intended or required in instances where such broadening phrases may be absent. Additionally, the various embodiments set forth herein are described in terms of exemplary schematics, block diagrams, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular configuration.

What is claimed is:

1. An insertion tool comprising:
an insertion portion having respective opposed proximal-end portion and distal-end portion, the insertion portion extends along a central axis for being inserted into a body; and
a drive power transmitting mechanism configured to be positioned on the proximal-end portion of the insertion portion for transmitting drive power to a driven member disposed on a distal-end side rather than the proximal-end portion,
wherein the drive power transmitting mechanism includes
a gear train being used for transmitting drive power from a first drive source that is electrically driven, and
a joint mechanism being used for joining the first drive source to the gear train so as to transmit the drive power from the first drive source to the gear train and unjoining the first drive source from the gear train; and
in a non-interlinked state in which the first drive source is unjoined from the gear train, a second drive source being joined to the joint mechanism to hold the gear train and the second drive source in an interlinked state.

2. The insertion tool of claim 1,
wherein the drive power transmitting mechanism is housed in a case disposed on the proximal-end portion of the insertion portion; and
the joint mechanism being capable to be exposed from the case.

3. The insertion tool of claim 1,
wherein the joint mechanism includes
a relay gear rotatable depending on the drive power from the first drive source, and
a hub engageable with the relay gear for bringing the first drive source and the gear train into the interlinked state and detachable from the relay gear for bringing the first drive source and the gear train into the non-interlinked state.

4. The insertion tool of claim 3,
wherein the gear train includes a rotational shaft disposed coaxially with a central axis of the relay gear;
the rotational shaft transmits the drive power from the first drive source to the gear train when the hub engages the relay gear to bring the first drive source and the gear train into the interlinked state; and
the relay gear idles with respect to the rotational shaft when the hub is detached from the relay gear to bring the first drive source and the gear train into the non-interlinked state.

5. An insertion system comprising:
the insertion tool of claim 1; and
the second drive source that is different from the first drive source, the second drive source being joined to the joint mechanism to bring the second drive source and the gear train into the interlinked state when the joint mechanism holds the first drive source and the gear train in the non-interlinked state.

6. The insertion system of claim 5, wherein the joint mechanism is capable of joining the first drive source to the joint mechanism for transmitting the drive power from the first drive source to the gear train, and of unjoining the first drive source from the joint mechanism.

7. The insertion system of claim 6, wherein
the first drive source joined to the joint mechanism; and
the second drive source that is manually driven and joined to the joint mechanism while the first drive source is being unjoined.

8. An insertion system of claim 6;
the first drive source joined to the joint mechanism; and
the second drive source that is electrically driven and joined to the joint mechanism while the first drive source is being unjoined.

9. The insertion system of claim 5, wherein the gear train makes a speed reduction in the drive power transmitted from the joint mechanism to the driven member.

10. A drive source used with the insertion tool of claim 1, comprising:
a joint portion that is able to be joined to the gear train,
wherein the drive power is able to be transmitted from the joint portion through the gear train to the driven member.

11. The drive source of claim 10, further comprising:
an electric motor supplied with electric power to generate the drive power transmitted from the joint portion through the gear train to the driven member.

12. The drive source of claim 10, further comprising:
a manual handle manually operable to generate the drive power transmitted from the joint portion through the gear train to the driven member.
